SEQUENCE LISTING (3 sequences)

SEQ ID NO: 1 - DNA, 1073 bp, Artificial sequence
  synthesized
aaatcccttataagaattctcacactggctcaccttcgggtgggcctttctgcgtttatatactagagagagaatataaaaagccagattattaatccggcttttttattatttaggtgatccatcgaactctagaatttgacggctagctcagtcctaggtacagtgctagcctagatactagagaaagaggagaaatactagatggttagcaaaggcgaagagctgatcaaggagaacatgcacatgaaactgtacatggaaggtaccgttaacaaccaccattttaagtgcaccagcgaaggcgagggtaaaccgtatgagggcacccagaccatgcgtatcaaggtggttgaaggtggcccgctgccgtttgcgttcgatattctggcgaccagctttatgtacggcagccgtaccttcatcaaccatacccagggtattccggatttctttaaacagagctttccggagggcttcacctgggaacgtgtgaccacctacgaggacggtggcgttctgaccgcgacccaggataccagcctgcaagacggttgcctgatctataacgtgaaaattcgtggcgttaactttccgagcaacggtccggtgatgcagaaaaagaccctgggctgggaagcgaacaccgagatgctgtacccggcggatggtggcctggaaggtcgtagcgacatggcgctgaagctggttggtggcggtcacctgatctgcaacttcaaaaccacctatcgtagcaaaaagccggcgaaaaacctgaagatgccgggcgtgtactatgttgatcatcgtctggaacgtattaaagaggcggacaaggaaacctacgtggaacagcacgaggtggcggttgcgcgttattgcgacctgccgagcaaactgggtcataagctgaactaaccaggcatcaaataaaacgaaaggctcagtcgaaagactgggcctttcgttttatctgttgtttgtcggtgaacgctctctactagagtcacactggctcaccttcgggtgggcctttctgcgtttataagctgggttaaccatcactgggatcc

SEQ ID NO: 2 - DNA, 4511 bp, Artificial sequence
  synthesized
tcgagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagtatgtcgacgccccagccatggttttagagctagaaatagcaagttaaaataaggctagtccgttatcaacttgaaaaagtggcaccgagtcggtgctttttttgaattctctagagtcgacctgcagaacatcaccgcaacgcaaatgatggagtcgatgatcaccaacccgatgccgacccgtgcggaagtgatggacgtggcgaacgccgtcctggatggcacggatgcggttatgctgtctgccgaaaccgcagccggtcagtatccttctgaaaccgttgccgcaatggcgcgcgtctgcctgggcgcagaaaaaatccccagcatcaatgtgtctaaacaccgtctcgacgtgcagttcgacaacgttgaagaagccattgccatgtctgcgatgtatgcggcaaaccatctgaaaggcgttaccgcgatcatcaccatgacggaatccggtcgtaccgcgctaatgacttcccgtatcagctccggcctgccgattttcgccatgtcgcgccatgaacgcacgctgaacctgaccgcgctctatcgcggagtaacgccggtgcattttgatagcgcggctgatggcgttgtcgcggcacatgaagctgttaatctgctgcgcgataaagggtatctggtttccggcgacctggttatcgtgacccagggcgatgtcatgagcaccgtcggttcaaccaataccacgcgcaaacgtccgccgaaacgccgacgcactgtgttccagatatagtcaaaaaccggattaccctgattatgaaacatcgccgccattttttgcccaaatcccttataagaattctcacactggctcaccttcgggtgggcctttctgcgtttatatactagagagagaatataaaaagccagattattaatccggcttttttattatttaggtgatccatcgaactctagaatttgacggctagctcagtcctaggtacagtgctagcctagatactagagaaagaggagaaatactagatggttagcaaaggcgaagagctgatcaaggagaacatgcacatgaaactgtacatggaaggtaccgttaacaaccaccattttaagtgcaccagcgaaggcgagggtaaaccgtatgagggcacccagaccatgcgtatcaaggtggttgaaggtggcccgctgccgtttgcgttcgatattctggcgaccagctttatgtacggcagccgtaccttcatcaaccatacccagggtattccggatttctttaaacagagctttccggagggcttcacctgggaacgtgtgaccacctacgaggacggtggcgttctgaccgcgacccaggataccagcctgcaagacggttgcctgatctataacgtgaaaattcgtggcgttaactttccgagcaacggtccggtgatgcagaaaaagaccctgggctgggaagcgaacaccgagatgctgtacccggcggatggtggcctggaaggtcgtagcgacatggcgctgaagctggttggtggcggtcacctgatctgcaacttcaaaaccacctatcgtagcaaaaagccggcgaaaaacctgaagatgccgggcgtgtactatgttgatcatcgtctggaacgtattaaagaggcggacaaggaaacctacgtggaacagcacgaggtggcggttgcgcgttattgcgacctgccgagcaaactgggtcataagctgaactaaccaggcatcaaataaaacgaaaggctcagtcgaaagactgggcctttcgttttatctgttgtttgtcggtgaacgctctctactagagtcacactggctcaccttcgggtgggcctttctgcgtttataagctgggttaaccatcactgggatcccaaaaaagccggcacacatcgcgtaccggctctgtcagcgcatttgttaatcgaagcgcagttgcggcagaacctctttcacctgtgccaggtattcacgacgatctgaccccgtcagaccttccgtgcgcggcaattttgctgtcagagggttaacggcttgctggttgatccatacctcataatgcagatgcggccctgtggaacgcccggtgttaccagaaagcgcaatacgatcgccacgtttcactttttgccccggtttcaccagcagcttacgcaagtgcatgtaacgtgtggtgtaggtgcgtccatgacgaatcgcaatgtagtaaccggcagcgccgctacgtttagcgaccacgacctcgccatcccccaccgacagcaccggcgtaccctgcggcatcgcaaagtcaacgccacgatgcggcgcaacgcgtccggtaaccgggttcagacgacgcggattgaagttggaggagatgcggaactgtttagcggtcgggaagcgtaaaaagcctttcgccagaccaaccccattacggtcatagaatttaccgtcagcggcgcgaatggcgtagtaagcttagatctattaccctgttatccctac

SEQ ID NO: 3 - DNA, 3851 bp, Artificial sequence
  synthesized
tcgagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgatgccgctcgccagtcgattggctgagctcatgaagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagtggcgctgttgcgcttcctgggttttagagctagaaatagcaagttaaaataaggctagtccgttatcaacttgaaaaagtggcaccgagtcggtgctttttttgaattctctagagtcgacctgcagaactcaactaccagaccaccgctttccacgataccgccaccgtccgtcagttgctgaatttacggccgtcgccggagtttgaacgctggctggaaacgatgggcattatggcaaacggtcgtctgaccaaacgggcgggcgatccgtcactgttcttctgatgacgcggggataacaccatggatcaaaaacagattgaagaaattgtacgtagcgtgatggcgtcaatgggacaggacgtaccgcagcccgccgcgccgtcaacgcaggaaggcgcaaagccgcagtgcgccgcgccgacggaaatcccttataagaattctcacactggctcaccttcgggtgggcctttctgcgtttatatactagagagagaatataaaaagccagattattaatccggcttttttattatttaggtgatccatcgaactctagaatttgacggctagctcagtcctaggtacagtgctagcctagatactagagaaagaggagaaatactagatggttagcaaaggcgaagagctgatcaaggagaacatgcacatgaaactgtacatggaaggtaccgttaacaaccaccattttaagtgcaccagcgaaggcgagggtaaaccgtatgagggcacccagaccatgcgtatcaaggtggttgaaggtggcccgctgccgtttgcgttcgatattctggcgaccagctttatgtacggcagccgtaccttcatcaaccatacccagggtattccggatttctttaaacagagctttccggagggcttcacctgggaacgtgtgaccacctacgaggacggtggcgttctgaccgcgacccaggataccagcctgcaagacggttgcctgatctataacgtgaaaattcgtggcgttaactttccgagcaacggtccggtgatgcagaaaaagaccctgggctgggaagcgaacaccgagatgctgtacccggcggatggtggcctggaaggtcgtagcgacatggcgctgaagctggttggtggcggtcacctgatctgcaacttcaaaaccacctatcgtagcaaaaagccggcgaaaaacctgaagatgccgggcgtgtactatgttgatcatcgtctggaacgtattaaagaggcggacaaggaaacctacgtggaacagcacgaggtggcggttgcgcgttattgcgacctgccgagcaaactgggtcataagctgaactaaccaggcatcaaataaaacgaaaggctcagtcgaaagactgggcctttcgttttatctgttgtttgtcggtgaacgctctctactagagtcacactggctcaccttcgggtgggcctttctgcgtttataagctgggttaaccatcactgggatccccgtcgaggccgacagaacctgtatttcaaacattcatcagggggggacgccgccagtagaagccgccgccgtgattgtggatttggccaaacggatgctggagcagaaagcgtccggcatcaacatgacccgttaaggagacatcatgcctgcattagatttaattcgaccttcagtgactgccatgcgcgtgattgcctccgtgaatgacggctttgcgcgggaacttaaattaccgccacatatacgtagtctcggactcatcacggcagattctgatgatgtgacctatattgccgctgagcttagatctattaccctgttatccctac

(19) United States
(12) Patent Application Publication
(10) Pub. No.: US 2024/0197792 A1
(43) Pub. Date: Jun. 20, 2024

(54) EFFICIENT TRACELESS GENE EDITING SYSTEM FOR SALMONELLA AND USE THEREOF

(71) Applicant: JIANGSU TARGET BIOMEDICAL RESEARCH INSTITUTE CO., LTD., Changzhou, Jiangsu (CN)

(72) Inventors: Zichun HUA, Changzhou, Jiangsu (CN); Jiahuang LI, Changzhou, Jiangsu (CN); Chao HAN, Changzhou, Jiangsu (CN); Menghui WANG, Changzhou, Jiangsu (CN); Junjie ZHOU, Changzhou, Jiangsu (CN); Jing LI, Changzhou, Jiangsu (CN)

(21) Appl. No.: 18/548,158

(22) PCT Filed: Oct. 29, 2020
(86) PCT No.: PCT/CN2020/124685

§ 371 Date: Aug. 28, 2023

(30) Foreign Application Priority Data
Mar. 16, 2020 (CN) 202010182038.0

(51) Int. Cl. A61K 35/74; A61P 35/00; C12N 9/12; C12N 9/22; C12N 15/11; C12N 15/74; C12N 15/90

(52) U.S. Cl. CPC A61K 35/74; A61P 35/00; C12N 9/1241; C12N 9/22; C12N 15/11; C12N 15/74; C12N 15/902; C12Y 207/07047; C12N 2310/20; C12N 2800/101; C12N 2800/40; C12N 2800/80

(57) ABSTRACT

An efficient and scarless gene editing system for Salmonella and the use thereof are provided. λRed recombinase is used to promote a double crossover of a DNA template with a genomic target site, the CRISPR/Cas9 system is used for screening, and one-step homologous recombination is used to rapidly construct a targeting plasmid, which can realize insertion, substitution or knock-out of genes. Genomic DNA is completely substituted according to a design of homologous template, and without other fragment residues. Genome editing can be completed efficiently within 3-4 days, thereby reducing the experimental workload and shortening the experimental cycle.

### INCORPORATED BY REFERENCE

The instant application contains a Sequence Listing which has been submitted electronically in ASCII format and is hereby incorporated by reference in its entirety. Said ASCII copy is named SequenceListingPCTCN2020124172.txt and is 13 kilobytes in size.

### FIELD OF THE INVENTION

The present invention relates to the field of genetic engineering, and specifically, to an efficient and traceless gene editing system for Salmonella and its use.

### BACKGROUND OF THE INVENTION

Salmonella is a common zoonotic pathogen and the most common pathogenic bacteria of bacterial food infections in countries, which can lead to a variety of syndromes such as gastroenteritis, typhoid fever, septicemia and extraintestinal focal infections. Modification of the Salmonella genome through genome editing technology to obtain strains with different genetic backgrounds can help study the mechanisms of Salmonella growth, reproduction and its pathogenic process, thereby laying a foundation for prevention and treatment. Attenuated Salmonella may also be used in tumor treatment, for example, it has been found that attenuated Salmonella strain VNP20009 has a certain therapeutic effect on a variety of tumors in animal models. Genetic modification of the Salmonella genome is an important means to further enhance its therapeutic effect, which can be achieved by modifying the genes related to invasion, motility or metabolism of the Salmonella genome, or by integrating therapeutic genes into the bacterial chromosome and with the stable expression thereof, to improve the therapeutic effect, or by integrating a reporter gene into the chromosome and with the stable expression thereof, to achieve in vivo tracing.

The λ phage-based Red homologous recombination system is a commonly used method for modifying bacterial genomes and has been used in Escherichia coli [Datsenko, K. A. 2000] and Salmonella [Husseiny M I et al, 2005; Solano C et al, 2010]. The genome after knock-out by the Red homologous recombination method will have a resistance gene screening marker, or a residual FRT (recombinase FLP recognition site) sequence trace of about 80 bp after removal of the marker [Datsenko, K. A. 2000]. The residual scarsequence may limit the use of the system for further modification of other genes. Recently, some traceless gene editing methods have been developed, such as the traceless editing systems based on I-Scel and λRed [Kim J et al. 2014; Blank K et al. 2011], however, these methods still have the drawbacks of cumbersome experimental process, long cycle time, and difficulty in large fragment insertion.

The adaptive immune systemCRISPR-Cas9 (clustered regularly interspaced short palindromic repeats/CRISPR-associated proteins), derived from Streptomyces, is an efficient gene editing tool that has emerged in recent years and has been applied to many eukaryotic cells and prokaryotic cells including E. coli, Streptomyces, Clostridium, and Lactobacillus [Jiang Y et al. 2015]. The CRISPR/Cas9 system comprises two components: sgRNA and nuclease Cas9. Targeting the target site of the genome is carried out by sgRNA through a specific 20-base sequence, and sgRNA mediates Cas9 cleavage of the site's DNA double strand, which can increase the proportion of homologous recombination or serve for screening. However, can this gene editing tool derived from Streptomyces be applied to Salmonella? If yes, what modifications should be made to facilitate genome editing in Salmonella? These problems have not been explored and studied.

Currently, there is a lack of a fast and efficient traceless gene editing system for Salmonella and its use.

### SUMMARY OF THE INVENTION

The objective of the present invention is to provide an efficient traceless gene editing system for Salmonella and use thereof.

In order to solve the technical problem identified above, the present invention provides the following technical solution: An efficient and traceless gene editing system for Salmonella, comprising: a Cas9 protein, a sgRNA, a λRed recombinase and a homologous recombinant DNA fragment, and vectors and gene sequences for carrying or expressing the same.

Further, the efficient and traceless gene editing system for Salmonella consists of a dual plasmid CRISPR/Cas9 system comprising a helper plasmid A expressing a relevant functional protein and a targeting plasmid B expressing a target site sgRNA.

Further, the helper plasmid A comprises nucleic acid sequences of components: a Cas9 protein, a λRed recombinase, a temperature-sensitive replicon, an sgRNA expression frame of the targeting plasmid B replicon, and a helper plasmid A screening marker gene, and wherein the recombinase and sgRNA are inducibly expressed.

Further, the targeting plasmid B comprises nucleic acid sequences of components: a replicon, a targeting plasmid screening marker gene, an sgRNA expression frame for a target site, and a DNA fragment for homologous recombination, and wherein the replicons of the helper plasmid A and the targeting plasmid B are capable of being replicated in Escherichia coli and Salmonella, the replicon and the screening marker gene of the plasmid B are different from the replicon and the screening marker gene of the plasmid A, and the replicon of the targeting plasmid B is compatible with the replicon of the helper plasmid A.

Further, the sgRNA expression frame has a promoter-(N)X-sgRNA backbone-terminator structure, and the target site DNA has a 5′-(N)X-NGG-3′ structure, and wherein (N)X denotes X Ns, N is any of the bases A, T, C or T, and X is an integer greater than 15 and less than 25.

Further, the X of the target site DNA is 20, and the homologous recombinant DNA fragment is upstream homology arm-insert fragment-downstream homology arm when used for knock-in or substitution, and upstream homology arm-downstream homology arm when used for knock-out; the DNA fragment is constructed in the targeting plasmid B or in a PCR product; and the gene editing system is capable of gene editing on multiple target sites simultaneously.

Further, the targeting plasmid B comprises editing modules for multiple target sites with a structure of plasmid backbone (resistance gene-replicon)-editing module 1 (target site 1 sgRNA expression frame-upstream homology arm 1-knock-in (or substitution) fragment-downstream homology arm 1)-editing module 2 (target site 2 sgRNA expression frame-upstream homology arm 2-knock-in (or substitution) fragment-downstream homology arm 2)-editing module n; however, the success rate of editing gradually decreases with the increase of target sites, and due to the time cost of plasmid construction, generally the number target sites is not greater than 3.

A method of constructing the targeting plasmid B of the present invention, comprises the following steps:
1) for knock-in (or substitution): amplifying the targeting plasmid B backbone, the target site sgRNA expression frame, the upstream/downstream homology arms, and the knock-in (or substitution) fragment, respectively, and ligating all DNA fragments using one-step homologous recombination;
2) for knock-out: amplifying the targeting plasmid B backbone, the target site sgRNA expression frame, and the upstream/downstream homology arms, respectively, and ligating all DNA fragments using one-step homologous recombination;
3) for targeting plasmid B contain in no homology arms, amplifying the upstream/downstream homology arms respectively, and ligating them as a linear DNA template.

Use of the efficient traceless gene editing system for Salmonella in genome editing, comprises the following steps:
1) introducing the helper plasmid A into Salmonella and inducing a λRed recombinase expression to prepare competent cells;
2) introducing the targeting plasmid B and a linear DNA template to the competent cells of step 1), or introducing a targeting plasmid B comprising the template DNA;
3) resuscitating the cells from step 2) and coating on a plate comprising two resistances corresponding respectively to the plasmid A and the plasmid B, and screening for positive clones with double crossover;
4) after performing PCR or sequencing verification of the positive clones, inducing sgRNA expression of the targeting plasmid B replicon and eliminating the plasmid B;
5) after verifying the elimination of the plasmid B, increasing the bacterial culture temperature and eliminating the plasmid A to obtain a successfully modified Salmonella clone.

Further, at the end of step 4), the plasmid A is retained, and step 2) is repeated to introduce the targeting plasmid B and a template DNA for targeting other loci.

Use of the efficient and traceless gene editing system for Salmonella in Salmonella genome editing.

Use of the efficient and traceless gene editing system for Salmonella in preparation of a Salmonella antitumor drug.

Use of the efficient and traceless gene editing system for Salmonella in preparation of an eutC gene-deficient Salmonella by genome editing.

Use of the genome edited eutC gene-deficient Salmonella in preparation of Salmonella antitumor drugs.

In one embodiment of the present invention, the plasmid A is pCas [6] and comprises: a constitutively expressed Cas9 protein, a λRed recombinase (three proteins, i.e. Exo, Beta and Gam), a promoter ParaB of λRed which is induced to be expressed by L-arabinose, a temperature-sensitive replicon repA101, a sgRNA expression frame of the targeting plasmid B replicon, a promoter Ptrc of sgRNA which is regulated by a lactose manipulator; a kanamycin resistance gene, and a lactose deterrent protein lacl.

In one embodiment of the present invention, the plasmid B is a pTAT plasmid, which is formed by ligating fragment 1 plasmid backbone (comprising: a pMB1 replicon, and an ampicillin resistance gene), fragment 2 target site sgRNA expression frame, and fragment 3 template DNA (homology arm upstream of the target site, exogenous insertion DNA, homology arm downstream of the target site). The order of ligation can be varied in practice).

Beneficial effects: With the CRISPR/Cas9 system, the present invention uses the target site (N) X-NGG sequence of the genome as the target site for reverse screening of wild-type Salmonella to establish an efficient, stable and traceless gene editing method for Salmonella. The method uses a constitutive promoter to continuously express Cas9 protein and the target site sgRNA, and continuously cuts the DNA double strand at the (N) X-NGG sequence of the wild-type bacteria, and the bacteria will not survive if they cannot or are too slow to repair the DNA damage. Only positive clones that the target site DNA is double-crossovered with the template DNA, either spontaneously or with the aid of the λRed recombinase, does not have the (N) X-NGG sequence, and can survive. The inducibly expressed sgRNA is also used to cleave the replicon DNA of plasmid B. This allows for rapid elimination of plasmid B and improves the efficiency of the operation.

### BRIEF DESCRIPTION OF THE DRAWINGS

The solution of the present invention will be better understood with Reference to the accompanying drawings, wherein:

FIG. 1 shows a schematic diagram of the design and construction of the targeting plasmid B pTAT-X-insert of the present invention; P1 to P10 are primers, up: upstream homology arm of the target site, down: downstream homology arm of the target site, X: target site sequence, insert: insert fragment; a is a schematic diagram of the design of the pTA plasmid and its primers; b is a schematic diagram of the design of the wild-type Salmonella genome target site and its primers; c is a schematic diagram of the design of the pTAT-X plasmid and its primers.

FIG. 2 shows a flowchart of the efficient and scarless gene editing system for Salmonella of the present invention.

FIG. 3 shows agarose gel electrophoresis of the PCR amplification products of the Salmonella VNP20009 with msbB locus substituted by RFP of the present invention, wherein 1-15 are Salmonella in the experimental group; V is control Salmonella; and M is DNA molecular weight Marker.

FIG. 4 shows the growth curve of the msbB strain of the present invention; pCas-VNP was used as a control. Three independent replicates, mean±SEM.

FIG. 5 shows the fluorescence intensity of the msbB strain of the present invention; pCas-VNP was used as a control. Three independent replicates, mean±SEM.

FIG. 6 shows the agarose gel electrophoresis of the PCR amplification products of the Salmonella VNP20009 with eutC locus substituted by RFP of the present invention; wherein, 1 to 10 are Salmonella in the experimental group; V is control Salmonella; and M is the DNA molecular weight Marker DL2000, with the bands being 2000, 1000, 750, 500, 250, and 100 bp, respectively.

FIG. 7 shows the growth curve of the eutC strain of the present invention; pCas-VNP was used as a control. Three independent replicates, mean±SEM.

FIG. 8 shows the fluorescence intensity of the eutC strain of the present invention; pCas-VNP was used as a control. Three independent replicates, mean±SEM.

FIG. 9 shows the respective tumor growth curves of the tumor-bearing mice in the eutC: RFP group, msbB::RFP group and PBS blank group of the present invention.

FIG. 10 shows the respective survival curves of the tumor-bearing mice in the eutC::RFP group, msbB::RFP group and PBS blank group of the present invention.

### DETAILED DESCRIPTION OF THE INVENTION

The present invention will be described in further detail below with reference to the accompanying drawings.

The accompanying drawings and Examples of the present invention are intended to illustrate specific embodiments of the present invention in greater detail so that the solutions of the present invention and the advantages thereof can be better understood, but not intended to limit the present invention.

The present invention will be further described below by specific Examples. The methods involved in the Examples are conventional technical means known to one skilled in the art, unless specified otherwise. The experimental methods involved in the Examples have been performed for a long period of time, and more than 6 effective numerical ranges have been tried for concentration, time and primer sequence. The Examples list only one of the parameters, which is not the only optional parameter. The reagents, materials, etc., involved in the Examples are commercially available, unless specified otherwise.

An efficient and traceless gene editing system for Salmonella, comprising: a Cas9 protein, a sgRNA, a λRed recombinase and a homologous recombinant DNA fragment, and vectors and gene sequences for carrying or expressing the same.

Further, the efficient and traceless gene editing system for Salmonella consists of a dual plasmid CRISPR/Cas9 system comprising a helper plasmid A expressing a relevant functional protein and a targeting plasmid B expressing a target site sgRNA.

Further, the helper plasmid A comprises nucleic acid sequences of components: a Cas9 protein, a λRed recombinase, a temperature-sensitive replicon, an sgRNA expression frame of the targeting plasmid B replicon, and a helper plasmid A screening marker gene, and wherein the recombinase and sgRNA are inducibly expressed.

Further, the targeting plasmid B comprises nucleic acid sequences of components: a replicon, a targeting plasmid screening marker gene, an sgRNA expression frame for a target site, and a DNA fragment for homologous recombination, and wherein the replicons of the helper plasmid A and the targeting plasmid B are capable of being replicated in Escherichia coli and Salmonella, the replicon and the screening marker gene of the plasmid B are different from the replicon and the screening marker gene of the plasmid A, and the replicon of the targeting plasmid B is compatible with the replicon of the helper plasmid A.

Further, the sgRNA expression frame has a promoter-(N)X-sgRNA backbone-terminator structure, and the target site DNA has a 5′-(N)X-NGG-3′ structure, and wherein (N)X denotes X Ns, N is any of the bases A, T, C or T, and X is an integer greater than 15 and less than 25.

Further, the X of the target site DNA is 20, and the homologous recombinant DNA fragment is upstream homology arm-insert fragment-downstream homology arm when used for knock-in or substitution, and upstream homology arm-downstream homology arm when used for knock-out; the DNA fragment is constructed in the targeting plasmid B or in a PCR product; and the gene editing system is capable of gene editing on multiple target sites simultaneously.

Further, the targeting plasmid B comprises editing modules for multiple target sites with a structure of plasmid backbone (resistance gene-replicon)-editing module 1 (target site 1 sgRNA expression frame-upstream homology arm 1-knock-in (or substitution) fragment-downstream homology arm 1)-editing module 2 (target site 2 sgRNA expression frame-upstream homology arm 2-knock-in (or substitution) fragment-downstream homology arm 2)-editing module n; however, the success rate of editing gradually decreases with the increase of target sites, and due to the time cost of plasmid construction, generally the number target sites is not greater than 3.

A method of constructing the targeting plasmid B of the present invention, comprises the following steps:
1) for knock-in (or substitution): amplifying the targeting plasmid B backbone, the target site sgRNA expression frame, the upstream/downstream homology arms, and the knock-in (or substitution) fragment, respectively, and ligating all DNA fragments using one-step homologous recombination;
2) for knock-out: amplifying the targeting plasmid B backbone, the target site sgRNA expression frame, and the upstream/downstream homology arms, respectively, and ligating all DNA fragments using one-step homologous recombination;
3) for targeting plasmid B contain in no homology arms, amplifying the upstream/downstream homology arms respectively, and ligating them as a linear DNA template.

Use of the efficient traceless gene editing system for Salmonella in genome editing,

comprises the following steps:
1) introducing the helper plasmid A into Salmonella and inducing a λRed recombinase expression to prepare competent cells;
2) introducing the targeting plasmid B and a linear DNA template to the competent cells of step 1), or introducing a targeting plasmid B comprising the template DNA;
3) resuscitating the cells from step 2) and coating on a plate comprising two resistances corresponding respectively to the plasmid A and the plasmid B, and screening for positive clones with double crossover;
4) after performing PCR or sequencing verification of the positive clones, inducing sgRNA expression of the targeting plasmid B replicon and eliminating the plasmid B;
5) after verifying the elimination of the plasmid B, increasing the bacterial culture temperature and eliminating the plasmid A to obtain a successfully modified Salmonella clone.

At the end of step 4), the plasmid A is retained, and step 2) is repeated to introduce the targeting plasmid B and a template DNA for targeting other loci.

Use of the efficient and traceless gene editing system for Salmonella in Salmonella genome editing.

Use of the efficient and traceless gene editing system for Salmonella in preparation of a Salmonella antitumor drug.

Use of the efficient and traceless gene editing system for Salmonella in preparation of an eutC gene-deficient Salmonella by genome editing.

Use of the genome edited eutC gene-deficient Salmonella in preparation of Salmonella antitumor drugs.

In one embodiment of the present invention, the plasmid A is pCas [6] and comprises: a constitutively expressed Cas9 protein, a λRed recombinase (three proteins, i.e. Exo, Beta and Gam), a promoter ParaB of λRed which is induced to be expressed by L-arabinose, a temperature-sensitive replicon repA101, a sgRNA expression frame of the targeting plasmid B replicon, a promoter Ptrc of sgRNA which is regulated by a lactose manipulator; a kanamycin resistance gene, and a lactose deterrent protein lacl.

In one embodiment of the present invention, the plasmid B is a pTAT plasmid, which is formed by ligating fragment 1 plasmid backbone (comprising: a pMB1 replicon, and an ampicillin resistance gene), fragment 2 target site sgRNA expression frame, and fragment 3 template DNA (homology arm upstream of the target site, exogenous insertion DNA, homology arm downstream of the target site). The order of ligation can be varied in practice).

### Example 1

### Materials and Methods

### 1. Strains and Culture Method

The strains and plasmids used in this Example are listed in Table 1. Among them, E. coli DH5a was used as the clone strain, and Salmonella VNP20009 was the strain to be modified. All strains were cultured in LB medium at 37° C., except for the strain containing the pCas plasmid, which was cultured at 30° C., unless specified otherwise. The strains were incubated in a stationary incubator or in a shaker with shaking at 220 rpm. Bacterial growth was measured using an Eppendorf spectrophotometer to determine the light absorption value at 600 nm (OD600).

LB medium formulation: tryptone 10 g/L, yeast extract 5 g/L, sodium chloride 10 g/L, solid medium with agar 15 g/L. Antibiotics were added to the medium as needed: kanamycin (Kan+) at a working concentration of 50 mg/L, ampicillin (Amp+) at a working concentration of 100 mg/L. L-arabinose and IPTG were used as inducers, and added to the medium at the concentrations provided in the following steps. The strains and plasmids used in the present invention are shown in Table 1.

TABLE 1

| Strains or | | |
|---|---|---|
| Plasmids | Properties | Source |
| Strains | | |
| DH5a | deoR endA1 gyrA96 hsdR17 (rk-mk+) recA1 | Vazyme |
| | relA1 supE44 thi-1 Δ(lacZYA-argF)U169 | |
| | Φ80lacZ ΔM15 F-λ- | |
| VNP20009 | msbB purI xyl EGTAr | ATCC |
| Plasmids | | |
| pET-22b(+) | AmpR | Novagen |
| pCas | repA101(Ts) kan Pcas-cas9 ParaB-Red laclq | [6] |
| | Ptrc-sgRNA-pMB1 | |
| pTargetF- | pMB1 aadA cadA-sgRNA | [6] |
| cadA | | |
| pTargetT- | pMB1 aadA msbB-sgRNA ΔmsbB::RFP | The present |
| msbB-RFP | | invention |
| pTAT- | pMB1 amp msbB-sgRNA ΔmsbB::RFP | The present |
| msbB-RFP | | invention |
| pTAT- | pMB1 amp eutC-sgRNA ΔeutC::RFP | The present |
| eutC2-RFP | | invention |

### 2. Reagent Materials

Plasmid extraction kit and agarose gel purification kit for PCR product purification were commercially available from TIANGEN. High-fidelity PCR enzyme pre-mix 2× Phanta Max Master Mix used for the fragments for plasmid construction, rapid PCR enzyme pre-mix 2× Rapid Taq Master Mix used for PCR identification, and homologous recombination one step cloning kit, i.e. ClonExpress MultiS One Step Cloning Kit, used for ligation of DNA fragments were all commercially available from Nanjing Vozyme Biotech Co., Ltd.

All reagents were commercially available.

### 3. PCR Conditions

Unless specified otherwise, the reaction system for obtaining a PCR product is shown in Table 2:

TABLE 2

| | | |
|---|---|---|
| 2 × Phanta Max Master Mix | 25 | μL |
| Primer F | 2 | μL |
| Primer R | 2 | μL |
| Template | 1 | μL |
| ddH₂O | 20 | μL |
| Total | 50 | μL |

PCR reaction conditions were 94° C. for 5 min, 30 cycles (94° C. for 15 s, 56-58° C. for 15 s, 72° C. for 5 min), 72° C. for 7 min, 16° C. Reaction time at 72° C. was calculated based on 30 s/kb.

The reaction system for PCR identification to check the length of DNA fragments is shown in Table 3.

TABLE 3

| | | |
|---|---|---|
| 2 × Rapid Taq Master Mix | 10 | μL |
| Primer F | 1 | μL |
| Primer R | 1 | μL |
| Template | 1 | μL |
| ddH₂O | 7 | μL |
| Total | 20 | μL |

PCR reaction conditions were 94° C. for 5 min, 30 cycles (94° C. for 15 s, 56-58° ° C. for 15 s, 72° C. for 5 min), 72° C. for 7 min, 16° C. Reaction time at 72° C. was calculated based on 15 s/kb.

### 4. DNA Ligation and Transformation.

DNA fragment homologous recombination and transformation conditions. The reaction system was prepared on ice as shown in Table 4 below.

TABLE 4

| Linearized vector | X | μL |
|---|---|---|
| n insertion fragments | Y₁ − Yₙ | μL |
| 5 × CE MultiS Buffer | 4 | μL |
| Exnase MultiS | 2 | μL |

| | |
|---|---|
| ddH₂O | To 20 μL |

The volume of each DNA fragment was calculated according to the ClonExpress MultiS One Step Cloning Kit Instruction Manual, the optimal amount for each fragment was [0.02×number of fragment base pairs] ng (0.03 pmol), the amount of linearized cloning vector should be between 50 ng and 200 ng, and the amount of each inserted fragment should be greater than 10 ng.

Recombination product was taken as 10 μL and added to 100 μLDH5a competent cells and placed on ice for 30 min. Heat-excited at 42° C. for 60 s and then immediately placed on ice to cool for 3 min. 900 μL of antibiotic-free LB medium was added and shaken at 37° C. for 1 h. Centrifuged at 5000 g for 5 min, and 900 μL supernatant was discarded. The bacteria were re-suspended in the remaining medium, and coated on a culture plate with correct resistance and subjected to an inverted incubation overnight at 37° C.

### 5. DNA Sequencing

The plasmids or bacteria solutions were sent to Nanjing Kingsley Biotechnology Co. Ltd (Kingsley) for DNA sequencing.

### 6. Salmonella Competence Preparation

The day before the experiment, Salmonella VNP20009 strain was inoculated in 3 ml LB medium and incubated overnight at 37° C. with shaking. 2) The next day, it was transferred to fresh LB medium and incubated with shaking, and when the OD600 was about 0.8, the bacteria were collected by centrifugation at 5000 g for 4 min. 3) The bacteria were washed with 10% glycerol for three times, and the number of bacteria was adjusted to 1×10⁹ CFU per tube, and finally, the bacteria were re-suspended in 80 μL of 10% glycerol.

### 7. Salmonella Electrotransformation

X μg plasmid was added to Salmonella VNP20009 competence, mixed fully, and then added to 2 mm Bio-Rad electroporation cup. 2) Gene Pulser Xcell™ electroporator was used for electroporation, with electroporation parameters set to 2400V, 25 μF, 4000. 3) After electrotransformation, resuscitated for 1 hr. in antibiotic-free LB at a suitable temperature, and then coated on aLB plate with corresponding resistance and subjected to an inverted incubation overnight at a suitable temperature.

### Example 2 Design and Construction of Gene Editing System

msbB modification site selection and sgRNA design. The full sequence data of the genome of Salmonella typhimurium strain VNP20009 was obtained from the NCBI database under the accession number CP007804.2. Part of the msbB gene between the pykA and yebA genes of Salmonella strain VNP20009 was missing. Firstly, we chose to insert a fragment between these two gene loci to substitute the 529 bp genomic sequence (CP007804.2, 1,886,524-1,887,052), which was not expected to change the characteristics of the strain. A sequence with a 5′-(N)_{X}-NGG-3′ structure was selected from the substituted sequence, with (N)_{X} denoting X N's, N being any of the bases A, T, C, or T, and X being a natural number greater than 15 and less than 25. In this Example, one sequence was selected: atgtcgacgccccagccatg, i.e., msbB-N20, and based on the selected N20 sequence, the primer pTargetF-msbB-F (sequence: atgtcgacgccccccagccatggttttagagagctagaaatagc) was designed to construct a targeting plasmid, thereby expressing sgRNA that can mediate Cas9 protein cleavage of the corresponding target site of msbB gene.

TagRFP-ORF design. Terminator B0014-promoter J23100-ribosome binding site B0034-TagRFP protein-terminator B0015, totaling 1073 bp, was synthesized by Kingsley. The specific sequence is shown as sequence 1 in the Sequence Listing.

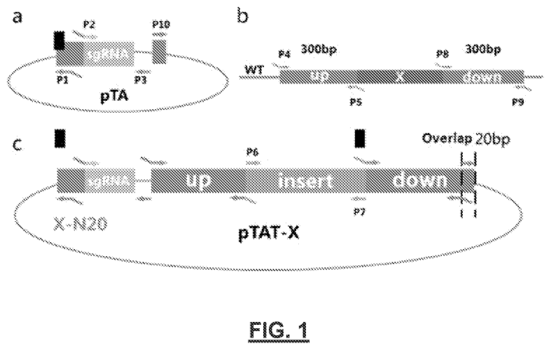

Plasmid BpTAT-X-insert design and construction (FIG. 1). Primers were designed (see Table 5), and the vector, target gene sgRNA, upstream homology arm of the target gene, insert fragment, and downstream homology arm of the target gene were amplified by PCR, respectively. The above DNA fragments have 15-20 bp homologous sequences between two of them, which can be used to link multiple fragments by one-step homologous recombination and thus shorten the experimental cycle.

The target gene sgRNA guides the Cas9 cleavage site target DNA sequence, which has a 5′-(N)_{X}-NGG-3′ structure. The method is designed without INSERT fragment. By directly connecting the upstream and downstream homology arms of the target gene, gene knock-out can be performed.

When the method is used to perform point mutation, genomic site mutation can be performed by designing primer PCR to obtain a homologous template carrying the mutation site. The primers used in this Example are shown in Table 5.

TABLE 5

| Primer | Sequence (5′-3′) | Use |
|---|---|---|
| PT V-F | atgccgctcgccagtcga | pTargetF vector |
| pT V-R | ctcgagtagggataacagggtaataga | |
| pT amp-F | ccctgttatccctactcgagttaccaatgcttaatcagtgaggc | AmpR |
| pT amp-R | aatcgactggcgagcggcatcgcggaacccctatttgtt | |
| pTargetF-msbB-R | catggctggggcgtcgacatactagtattatacctaggac | pTA vector |
| pTA-vector-R | gcttagatctattaccctgttatcc | |
| pTargetF-msbB-F | atgtcgacgccccagccatggttttagagctagaaatagc | msbB-sgRNA |
| pTA-vector-F | ttctgcaggtcgactctagagag | |
| pTA-RFP on-F | tctagagtcgacctgcagaacatcaccgcaacgcaaat | Upstream homology arm of |
| RFP-msbB-up-R | tttgggcaaaaaatggcggcg | msbB |
| rfp-msbb-mid-f | gccgccattttttgcccaaatcccttataagaattctcacactgg | RFP-ORF |
| RFP-msbB-Mid-R | ggcttttttgggatcccagtgatggttaaccc | |
| rfp-msbb-lower-f | actgggatcccaaaaaagccggcacacatc | downstream homology arm of |
| pTA-RFP down-R | acagggtaatagatctaagcttactacgccattcgcgc | msbB |
| msbB-N20 | atgtcgacgccccagccatg | Identification |
| pTA-PCR | ctctagcttcccggcaac | PTAT-msbB-RFP |
| identification-R | | |

pTA vector construction. The genome of Salmonella VNP20009 strain is with aadA gene expressing streptomycin adenylyltransferase, and it is necessary to substitute the aadA resistance gene of pTargetF with a suitable resistance gene as a screening marker. The pTargetT-msbB-RFP constructed in this experiment (sequence not shown) could not work in Salmonella VNP20009, indicating that not all plasmids designed and constructed can work, but require experimental screening. The pTargetF resistance gene, aadA, was substituted with AmpR. Primers pT V-F/pT V-R were used for vectorPCRamplification with pTargetF as the template, and primers pT amp-F/pT amp-R were used for AmpR PCR amplification with pET-22b(+) as the template. The above two linear DNA fragments were recovered and purified from the gels, and then subjected to ligation at 37° C. for 30 min by one-step homologous recombination (based on the DNA ligation conditions). The recombinant products after DH5a transformation were taken and coated to an Amp+-resistant plate and subjected to inverted incubation overnight at 37° C. Monoclones were selected and verified.

Construction of pTAT-msbB-RFP plasmid (see sequence 2 in the Sequence Listing). The construction method is shown in FIG. 1. Primers pTargetF-msbB-R (P1)/pTA-vector-R (P10) were used for pTAT vector backbone PCR amplication with the pTA plasmid as the template; primers pTargetF-msbB-F (P2)/pTA-vector-F (P3) were used for msbB-sgRNA amplication with the pTA plasmid as the template; primers pTA-RFP-up-F (P4)/RFP-msbB-up-R (P5) were used for amplification of the upstream homology arm 678 bp of the msbB locus with the Salmonella VNP20009 genome as the template; primers RFP-msbB-middle-F (P6)/RFP-msbB-middle-R (P7) were used for RFP-ORF amplication with the synthetic TagRFP fragment as the template; primers RFP-msbB-down-F (P8)/pTA-RFP-down-R (P9) were used for amplification of the downstream homology arm 587 bp of the msbB locus with the Salmonella VNP20009 genome as the template. The above five linear DNA fragments were recovered and purified from the gels, and then subjected to ligation at 37° C. for 30 min by one-step homologous recombination (based on the DNA ligation conditions). The recombinant products after DH5a transformation were taken and coated to an Amp+-resistant plate and subjected to inverted incubation overnight at 37° C. Monoclones were selected and verified. The verification primer was msbB-N20/pTA-PCR identification-R, and the positive clone PCR band was 2770 bp.

### Example 3 Substitution of the Salmonella VNP20009 msbB Locus with the RFP Gene

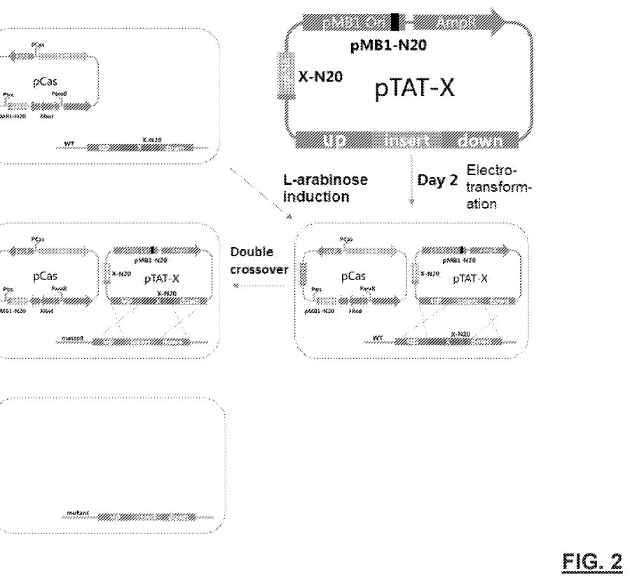

The flowchart for the application of the efficient and traceless Salmonella gene editing system is shown in FIG. 2. On the first day pCas plasmid is transferred to Salmonella, and the resulting strains from this step can be conserved for editing of a series of target genes. On the second day, the pCas-Salmonella competence is prepared by adding L-arabinose to induce expression of the A-Red recombinase, followed by transfection of the pTAT-X plasmid and coating of Kan+/Amp+ plates. The homologous template in the positive clone is double crossover with the target gene, and the wild-type clone is subjected to X-N20 sgRNA-mediated Cas9 sustained cleavage of the double-stranded DNA, resulting in difficulty in growth of the wild strain. On the third day, monoclones are picked to identify the effect of genome editing, and the clones with successful editing are added to IPTG to induce pMB1-N20 sgRNA expression targeting the replicon of the pTAT-X plasmid, and the pTAT plasmid was removed. If a multi-gene modification is required, the strain obtained in this step contains the pCas plasmid and can be further used as a host bacteria for other gene modifications for a new round of editing. On the fourth day, the pCas plasmid is removed by incubation at 42° C. The primers used in this Example are shown in Table 6:

TABLE 6

| Primer | Sequence (5′-3′) |
|---|---|
| pCas- | cagcttacatggcccaggtg |
| identification-F | |
| pCas- | aattcgtgtcgctcaaggcg |
| identification -R | |
| msbB-RFP- | gcgatccggagctggttg |
| identification-F | |
| msbB-RFP- | gcgagatgctggatggca |
| identification-R | |

pCas plasmid electrotransformed Salmonella VNP20009. Salmonella VNP20009 competence was prepared as described in Materials and Method 6. 1 μg of pCas plasmid was added to Salmonella VNP20009 competence, and after electrotransformation, it was resuscitated in antibiotic-free LB at 30° C. for 1 hour, then coated on Kan+-resistant LB plate, and subjected to inverted incubation at 30° C. overnight. Monoclones were picked and incubated at 30° C. to preserve the seed, and pCas-VNP20009 (pCas-VNP) strain was obtained. The verification primers were pCas-identification-F/pCas-identification-R, and the positive colony band containing pCas plasmid was 765 bp.

Preparation of Salmonella pCas-VNP20009 competence. 1) The Salmonella pCas-VNP strains obtained by electrotransformation were selected, plate streaking in Kan+-resistant LB, and incubate at 30° C. overnight. 2) The monoclines were picked and incubated in Kan+LB medium with shaking, and arabinose (10 mM final concentration) was added when OD₆₀₀ was about 0.2-0.3 to induce expression of Red recombinase. 3) When OD₆₀₀ was about 0.6-0.8, centrifuged at 5000 g for 4 min to collect the bacteria. 4) Washed with 10% glycerol for 3 times, the number of bacteria in each tube was adjusted to 1×10⁹ CFU, and finally the bacteria were resuspended in 80 μL of 10% glycerol.

The pTAT-msbB-RFP plasmid electrotransformed Salmonella pCas-VNP. 1 μg pTAT-msbB-RFP plasmid was added to Salmonella pCas-VNP competence, and after electrotransformation, it was resuscitated for 1 hr at 30° C. in antibiotic-free LB, coated on Kan+/Amp+-resistant LB plates, and then subjected to inverted incubation at 30° C. overnight.

msbB site RFP substitution verification. A number of monoclonal clones on the above Kan+/Amp+-resistant LB plates, and Salmonella VNP20009 wild type (WT) clone was used as a negative control, and the colonies were subjected to agarose gel electrophoresis after PCR using the primers msbB-RFP-identification-F/msbB-RFP-identification-R. The positive band for successful knock-in was 2475 bp and the negative band for unsuccessful knock-in or WT was 1931 bp. Editing efficiency=number of positive colonies/total number of colonies×100%.

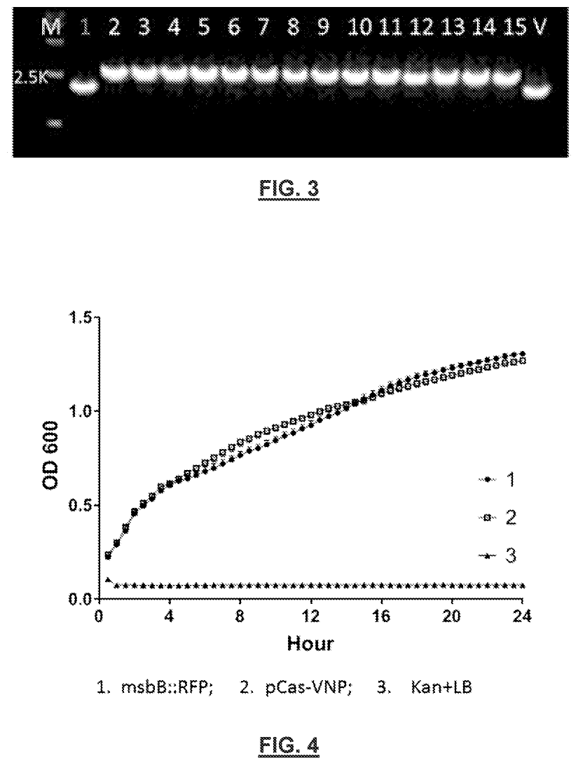

The results of the msbB site RFP substitution are shown in FIG. 3. 14 of the 15 monoclones were knocked in successfully and 1 failed, with an editing efficiency being 93.3%.

Elimination of pTAT plasmid. The clones verified to be knocked-in successfully were transferred to 3 ml Kan+LB medium, and IPTG (final concentration 0.5 mM) was added and incubated at 30° C. for 8 h. The sgRNA expression targeting the pTAT replicon was induced, and in combination with Cas9 to cause the DNA double-strand break at the PTAT plasmid replicon. The cultured bacteria solutions on the Kan+/Amp+-resistant LB plate and Kan+-resistant LB plates were streaked. The pTAT plasmid has been eliminated if no Salmonella clone grows from the double-resistant plate, and if it is found that some Salmonella clones grow, it is again transferred to a Kan+LB medium containing IPTG. Some monoclones were selected from the Kan+-resistant plate and blown and mixed well in 10 μL of sterile water, and 5 μL was added to Kan+LB and Kan+/Amp+LB respectively. pTAT plasmid elimination was confirmed by the growth of Salmonella clones only in Kan+LB. Several Salmonella monoclones grown on Kan+ plates after IPTG induction can also be directly identified by PCR, e.g., using the primer pTA-PCR identification-R/RFP-msbB-down-F, and using Salmonella clones without elimination of pTAT as a positive control. If no band is identified by PCR, it indicates that the pTAT plasmid has been removed.

Elimination of pCas plasmid. The Salmonella clones after elimination of the pTAT plasmid were transferred to the antibiotic-free LB and incubated at 42° C. for 8 h. The bacteria solution was streaked on the antibiotic-free LB plate, and Salmonella monoclones were selected and transferred to the Kan+ or antibiotic-free LB, and the Salmonella clones that did not grow in the Kan+LB were considered to be successful in elimination of pCas.

Bacterial growth curve and fluorescence assay. Three clones with pTAT plasmid eliminated and three Salmonella clones containing pCas were selected and transferred to 3 mL Kan+LB and incubated overnight. 300 μL of Kan+LB medium was added to each well of the culture plate for Bioscreen automatic growth curve analyzer, and then 1 μL of the overnight culture solution was added, and incubate at 30° C. for 24 hr. The overnight culture solution was transferred to a 3 mL Kan+LB tube in a volume ratio of 1/1000 and incubated at 30° C. for 24 hr. The fluorescence intensity of RFP (excitation wavelength 550 nm, emission wavelength 590 nm) and OD₆₀₀ were measured by using TECAN Infinite M200 microplate reader.

The growth curve of Salmonella msbB strain is shown in FIG. 4, and there is no significant difference between the growth curves of the successfully edited Salmonella clone msbB::RFP and Salmonella pCas-VNP, which indicates that the efficient and traceless Salmonella gene editing system of the present invention can achieve accurate positioning and design, and is free of off-target that may occur in CRISPR-Cas9. Three independent replicates, MEAN±SEM.

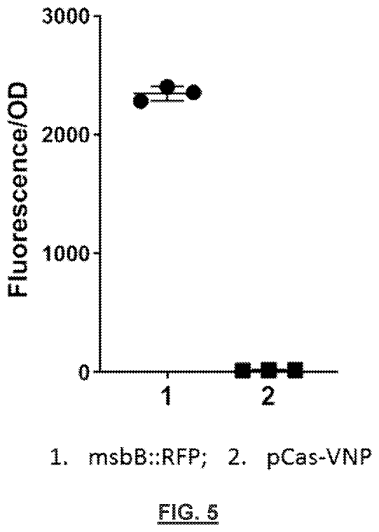

The fluorescence intensities of the Salmonella msbB strains are shown in FIG. 5. Significant fluorescence was detected for the successfully edited Salmonella clone msbB::RFP, and the fluorescence of Salmonella VNP20009 was essentially equivalent to that of the LB background. Fluorescence was calculated as (fluorescence-fluorescence_{LB})/(OD-OD_{LB}). Three independent replicates, mean±SEM.

### Example 4 Substitution of the Salmonella VNP20009 eutC Locus with the RFP Gene

pTAT-eutC-RFP plasmid was constructed (see sequence 3). 636 bp in the eutC-ORF (CP007804.2, 2,508,639˜ 2,509,274) was substituted with RFP-ORF. 5′-ggcgctgttgcgcttcctgg-3′ was selected as eutC2-N20, and based on the selected N20 sequence, the primer pTA-eutC2-F (sequence: ggcgctgttgcctgccttcctgggttttagagagctagaaatagc) was designed to construct a targeting plasmid, so that sgRNA that can mediate Cas9 protein cleavage of the corresponding target sites of the msbB gene is expressed. Primers pTA-eutC2-R/pTA-vector-R were used for PCR amplification of the pTAT vector backbone, with the pTA plasmid as the template; primers pTA-eutC2-F/pTA-vector-F were used for amplification of the eutC2-sgRNA, with the pTA plasmid as the template; primers eutC2-plasmid template up-F/eutC2-up RFP-R were used for amplification of the upstream homology arm 301 bp of the eutC locus, with the Salmonella VNP20009 genome as the template; primers RFP-msbB-mid-F/RFP-msbB-mid-R were used for amplification of RFP-ORF, with the synthesized TagRFP fragment as the template; and primers eutC2-down RFP-F/eutC2-plasmid-template down-R were used for amplification of the downstream homology arm 304 bp of the eutC locus, with Salmonella VNP20009 genome as the template. The ligation step was the same as in Example 1. Verification primer was eutC2-N20/pTA-PCR identification-R, and the positive clone PCR band was 2110 bp. The primers used in this Example are shown in Table 7.

TABLE 7

| Primer | Sequence (5′-3′) | Use |
|---|---|---|
| pTA-eutC2-R | ccaggaagcgcaacagcgccactagtattatacctaggac | pTA vector |
| pTA-vector-R | gcttagatctattaccctgttatcc | |
| pTA-eutC2-F | ggcgctgttgcgcttcctgggttttagagctagaaatagc | eutC2-sgRNA |
| pTA-vector-F | ttctgcaggtcgactctagagag | |
| eutC2-plasmid template | tctagagtcgacctgcagaactcaactaccagaccaccgc | eutC upstream |
| upper-F | | homology arm |
| eutC2-upper RFP-R | agaattcttataagggatttccgtcggcgcggcgcgcact | |
| rfp-msbb-mid-f | gccgccattttttgcccaaatcccttataagaattctcacactgg | RFP-ORF |
| RFP-msbB-mid-R | ggcttttttgggatcccagtgatggttaaccc | |
| eutC2-lower RFP-F | gttaaccatcactgggatccccgtcgaggccgacagaa | eutC downstream |
| eutC2-plasmid template | acagggtaatagatctaagctcagcggcaatataggtcac | homology arm |
| lower-R | | |
| eutC2-N20 | ggcgctgttgcgcttcctgg | appraise |
| pTA-PCR identification-R | ctctagcttcccggcaac | PTAT-eutC2-RFP |
| eutC-RFP-identification-F | gtgaacaccgtggtgggc | Identification of |
| eutC-RFP-identification-R | gcgcgatacctgcggtag | eutC:: RFPs |
| | | Substitution results |

The method of Preparation of Salmonella pCas-VNP competence was as described in Example 2.

The pTAT-eutC-RFP plasmid electrotransformation of Salmonella pCas-VNP was as described in Example 2.

Verification of eutC siteRFP substitution. The method was as described in Example 2. Primers eutC-RFP-identification-F/eutC-RFP-identification-R were used, and the positive band for successful knock-in was 1953 bp, and the negative band for unsuccessful knock-in or wild-type was 1750 bp.

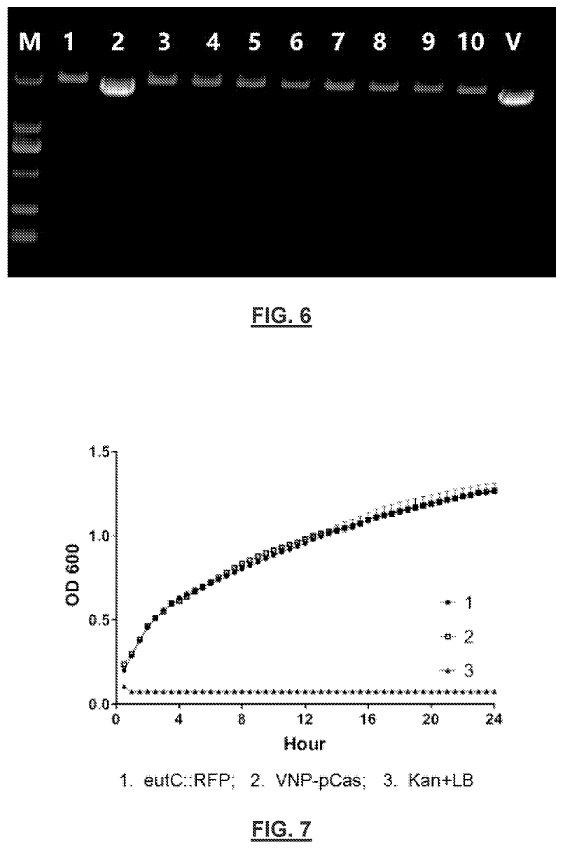

The results of RFP substitution for the eutC locus are shown in FIG. 6, with 9 out of 10 monoclones knocked in successfully and 1 failed, editing efficiency 90%.

The methods of eliminating the pTAT-eutC-RFP plasmid and the pCas plasmid were as described in Example 2.

The growth curve of eutC strain are shown in FIG. 7. There is no significant difference between the growth curves of the successfully edited Salmonella clone eutC::RFP and Salmonella pCas-VNP, which indicates that the efficient and traceless Salmonella gene editing system of the present invention can achieve accurate positioning and design, and is free of off-target that may occur in CRISPR-Cas9. Three independent replicates, MEAN±SEM.

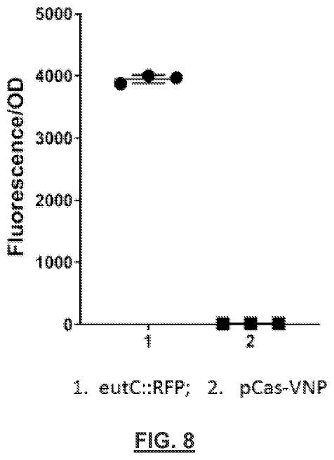

The fluorescence intensity of the eutC strains are shown in FIG. 8. Significant fluorescence was detected for the successfully edited Salmonella clone eutC::RFP, and the fluorescence of Salmonella VNP20009 was essentially equivalent to that of the LB background. Fluorescence was calculated as (fluorescence-fluorescence_{LB})/(OD-OD_{LB}). Three independent replicates, mean±SEM.

### Example 5 Evaluation of Gene-Edited Salmonella VNP20009 eutC for Antitumor Applications

The B16F10 melanoma mouse model was constructed. B16F10 mouse melanoma cells were grown in DMEM cell culture medium until the exponential growth phase and then digested with 0.5% pancreatin, followed by centrifugation at 1000 rpm/min for 3 min, and the supernatant culture medium was removed and washed twice with PBS for cell counting, and the cells were finally resuspended in PBS to adjust the final concentration of cells to 2×10⁶ cells/mL. Each C57BL/6 mouse was inoculated with 100 μL of cells at the axillary fat pad of the mouse, i.e., 2×10⁵ cells per mouse. After inoculation, the mice were raised in a clean-grade animal house, and the subsequent experiments were performed when the tumor volume of the mice grew to approximately 150 mm³.

The anti-tumor effect of the modified strains were evaluated. Plasmid-free Salmonella VNP20009-msbB::RFP (msbB) and Salmonella VNP20009-eutC::RFP (eutC) strains were subjected to plate streaking on an antibiotic-free LB plate and incubated overnight at 37° C., and the Salmonella monoclones were picked off and transferred to 3 mL of LB for resuscitation, and then transferred to 3 ml of LB and incubated until the OD value was about 0.8, and subsequently centrifuged at 5000 g for 3 min to collect the bacteria, washed twice with sterile PBS, and then resuspended in PBS to adjust the final concentration to 1×10⁶ cfu/mL. 22 tumor-bearing mice were then taken and randomly divided into 3 groups (6 mice in the PBS group, and 8 mice in the msbB or eutC groups), and the mice in the msbB and eutC groups were intraperitoneally injected with 100 μL of the Salmonella msbB strains and Salmonella eutC strains, i.e., 1×10⁵ cfu, respectively. The PBS group was intraperitoneally injected with 100 μL of PBS. Tumor size and survival status of the mice were recorded continuously.

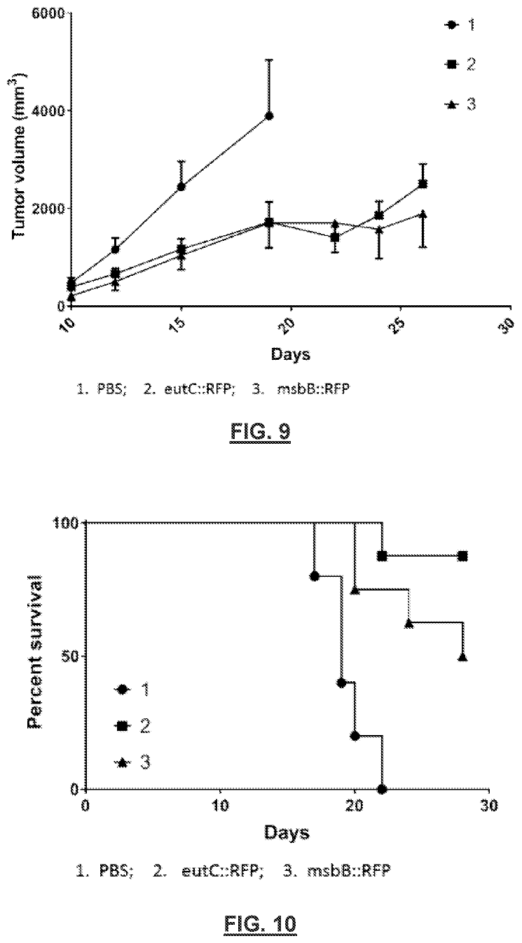

The tumor growth curves of the tumor-bearing mice are shown in FIG. 9. There was no significant difference in tumor size between the Salmonella eutC group and the Salmonella msbB group, indicating that lack of the eutC gene had no significant effect on the antitumor effect of Salmonella VNP20009, which verified the results of the bacterial growth in vitro. However, the Salmonella experimental group had a significant antitumor effect compared to the negative PBS control.

The survival curves of the tumor-bearing mice are shown in FIG. 10. Mice in the Salmonella eutC group had significantly longer survival time than the Salmonella msbB group (P<0.05), and both Salmonella groups had significantly longer mouse survival time than the PBS group. The results of significant prolongation of survival time of mice in the Salmonella eutC group compared to the Salmonella msbB group are significantly different from the results of in vitro bacterial growth as well as from the results of FIG. 9, indicating that the Salmonella eutC produced by the technology of the invented efficient and traceless gene editing system for Salmonella has a promising application in the preparation of antitumor drugs.

Utilizing the technology of an efficient and traceless gene editing system for Salmonella of the present invention, 8 different target sites/target genes evenly distributed on the chromosome around the replication initiation site of the Salmonella chromosome were selected, which are located at positions 245,209, 1,290,176, 1,886,549, 2,509,349, 3,299,845, 3,988,068, 3,996,444, 4,483,494 bp of the bacterial chromosome, and the editing efficiencies were all above 90%, thereby systematically verifying the high efficiency of the invented efficient and traceless gene editing system for Salmonella in Salmonella gene editing.

The above description shows and describes the basic principle, main features and advantages of the present invention. One skilled in the art should understand that the present invention is not limited by the above embodiments, and what is described in the above embodiments and the specification is only to illustrate the principle of the present invention, and there will be various changes and improvements to the present invention without departing from the spirit and scope thereof. The scope of the present invention is defined by the appended claims, specification and the equivalents thereof.

What is claimed is:

1. An efficient and traceless gene editing system for Salmonella, wherein the efficient and traceless gene editing system for Salmonella consists of a dual plasmid CRISPR/Cas9 system comprising a helper plasmid A expressing a relevant functional protein and a targeting plasmid B expressing a target site sgRNA.

2. The efficient and traceless gene editing system for Salmonella according to claim 1, wherein the helper plasmid A comprises nucleic acid sequences of components: a Cas9 protein, a λRed recombinase, a temperature-sensitive replicon, an sgRNA expression frame of the targeting plasmid B replicon, and a helper plasmid A screening marker gene, and wherein the recombinase and sgRNA are inducibly expressed.

3. The efficient and traceless gene editing system for Salmonella according to claim 2, wherein the targeting plasmid B comprises nucleic acid sequences of components: a replicon, a targeting plasmid screening marker gene, an sgRNA expression frame for a target site, and a DNA fragment for homologous recombination, and wherein the replicons of the helper plasmid A and the targeting plasmid B are capable of being replicated in Escherichia coli and Salmonella, the replicon and the screening marker gene of the plasmid B are different from the replicon and the screening marker gene of the plasmid A, and the replicon of the targeting plasmid B is compatible with the replicon of the helper plasmid A.

4. The efficient and traceless gene editing system for Salmonella according to claim 3, wherein the sgRNA expression frame has a promoter-(N)X-sgRNA backbone-terminator structure, and the target site DNA has a 5′-(N)X-NGG-3′ structure, and wherein (N)X denotes X Ns, N is any of the bases A, T, C or T, and X is an integer greater than 15 and less than 25.

5. The efficient and traceless gene editing system for Salmonella according to claim 4, wherein the X of the target site DNA is 20, and the homologous recombinant DNA fragment is upstream homology arm-insert fragment-downstream homology arm when used for knock-in or substitution, and upstream homology arm-downstream homology arm when used for knock-out; the DNA fragment is constructed in the targeting plasmid B or in a PCR product; and the gene editing system is capable of gene editing on multiple target sites simultaneously.

6. The efficient and traceless gene editing system for Salmonella according to claim 5, wherein the targeting plasmid B comprises editing modules for multiple target sites with a structure of plasmid backbone (resistance gene-replicon)-editing module 1 (target site 1 sgRNA expression frame-upstream homology arm 1-knock-in (or substitution) fragment-downstream homology arm 1)-editing module 2 (target site 2 sgRNA expression frame-upstream homology arm 2-knock-in (or substitution) fragment-downstream homology arm 2)-editing module n, and the number target sites is not greater than 3.

7. A method of constructing the targeting plasmid B, comprises the following steps:
1) for knock-in (or substitution): amplifying the targeting plasmid B backbone, the target site sgRNA expression frame, the upstream/downstream homology arms, and the knock-in (or substitution) fragment, respectively, and ligating all DNA fragments using one-step homologous recombination; or
2) for knock-out: amplifying the targeting plasmid B backbone, the target site sgRNA expression frame, and the upstream/downstream homology arms, respectively, and ligating all DNA fragments using one-step homologous recombination; or
3) for targeting plasmid B contain in no homology arms, amplifying the upstream/downstream homology arms respectively, and ligating them as a linear DNA template.

8. A use of the efficient traceless gene editing system for Salmonella in genome editing, comprises the following steps:
1) introducing the helper plasmid A into Salmonella and inducing a λRed recombinase expression to prepare competent cells;
2) introducing the targeting plasmid B and a linear DNA template to the competent cells of step 1), or introducing a targeting plasmid B comprising the template DNA;
3) resuscitating the cells from step 2) and coating on a plate comprising two resistances corresponding respectively to the plasmid A and the plasmid B, and screening for positive clones with double crossover;
4) after performing PCR or sequencing verification of the positive clones, inducing sgRNA expression of the targeting plasmid B replicon and eliminating the plasmid B;
5) after verifying the elimination of the plasmid B, increasing the bacterial culture temperature and eliminating the plasmid A to obtain a successfully modified Salmonella clone.

9. The use of the efficient traceless gene editing system for Salmonella in genome editing according to claim 8, wherein at the end of step 4), the plasmid A is retained, and step 2) is repeated to introduce the targeting plasmid B and a template DNA for targeting other loci.

10. (canceled)

11. The use of the efficient traceless gene editing system for Salmonella in genome editing according to claim 8, wherein the successfully modified Salmonella is an eutC gene-deficient Salmonella.

12. The use of the efficient traceless gene editing system for Salmonella in genome editing according to claim 8, wherein the eutC gene-deficient Salmonella is used to prepare Salmonella antitumor drugs.

13. (canceled)
